(19) United States
(12) Patent Application Publication
(10) Pub. No.: US 2007/0207516 A1
(43) Pub. Date: Sep. 6, 2007

(54) Cholesterol measurement system and method

(76) Inventor: Thomas H. Doupe, Huntington Beach, CA (US)

Correspondence Address: DLA PIPER RUDNICK GRAY CARY US, LLP, 2000 UNIVERSITY AVENUE, E. PALO ALTO, CA, 94303-2248, US

(21) Appl. No.: 11/316,520

(22) Filed: Dec. 21, 2005

(51) Int. Cl. C12Q 1/60; G06G 7/48; G06F 19/00

(52) U.S. Cl. 435/11; 703/11; 702/19

(57) ABSTRACT

A cholesterol testing system and method are provided in which cholesterol values determined from testing a sample are analyzed and normalized to generate a set of cholesterol values.

### FIELD OF THE INVENTION

The invention relates generally to a system and method for determining cholesterol and in particular to a computer-implemented system for determining cholesterol.

### BACKGROUND OF THE INVENTION

It is well known that it is desirable to accurately measure the levels of certain types of cholesterols in a patient since certain types of cholesterol are strongly correlated with atherosclerosis and coronary heart disease. More recent studies also indicate that specific fractions of cholesterol are more closely associated with coronary heart disease than others. Recent studies have implicated LDL (low density lipoprotein) as the class of lipoprotein responsible for the accumulation of cholesterol in cells, whereas HDL (high density lipoprotein) has been shown to be important in the removal of excess cholesterol from cells. Thus, increased levels of LDL cholesterol have been associated with the greater risk of coronary heart disease, while a strong inverse relationship exists between HDL cholesterol and the risk of coronary heart disease.

In addition to LDL and HDL, several other lipoproteins have been shown to represent independent risk factors for coronary heart disease. Increased plasma concentrations of lipoprotein(a) [Lp(a)], a cholesterol rich lipoprotein, has been observed in survivors of myocardial infarction. One study, which reports the relationship of levels of Lp(a) and coronary heart disease in patients who underwent coronary angiography, concluded that plasma Lp(a) appears to be a major independent risk factor for coronary heart disease. Elevation of plasma VLDL is seen in survivors of myocardial infarction, suggesting the possible involvement of this lipoprotein in the atherosclerotic process.

The measurement of total cholesterol alone may not be adequate to identify subjects at risk for coronary heart disease. An individual with normal or near normal levels of total cholesterol may still be at risk because of low HDL levels, elevated Lp(a) levels, or elevated levels of VLDL. Moreover, the predictive power of total cholesterol for risk of coronary heart disease diminishes in men with increasing age. Therefore, assessment of the distribution of cholesterol among all the lipoproteins (a lipoprotein cholesterol profile), in addition to total cholesterol, is desirable in order to accurately assess risk for coronary heart disease.

Methods currently used to determine the concentration of cholesterol in the different lipoprotein classes can be divided into direct methods and indirect methods. In direct methods, lipoprotein cholesterol is determined by enzymatic assay of the individual lipoproteins, which are separated by ultracentrifugation, electrophoresis, or selective precipitation. The most accurate of these methods involves ultracentrifugation. However, ultracentrifugation separation methods are expensive, time-consuming, and are not practical for clinical applications wherein multiple analyses are carried out in large numbers.

Another method of determination of cholesterol distribution among plasma lipoproteins involves the separation of lipoproteins by high performance liquid chromatography and the on-line detection of cholesterol in the postcolumn effluent using an enzymatic reagent. This method also provides a direct measure of lipoprotein cholesterol. However, this method requires a relatively long retention period of separation of the sample. Moreover, the separation technique results in some loss of lipoproteins which could result in an underestimation of cholesterol concentration.

Indirect methods, as a general rule, are better suited for clinical applications than are direct methods. The most commonly used method for measurements of lipoprotein cholesterol performs multiple analyses using different aliquots of the same plasma sample. Total cholesterol (TC) is measured using a first aliquot of the sample. In a second aliquot, VLDL and LDL are removed by precipitation and the supernatant is assayed for cholesterol to provide a measure of HDL cholesterol. An estimate of LDL is obtained by measuring the triglycerides (TG) in a third aliquot using the Friedewald formula or is measured directly after ultracentrifugal isolation of very low density lipoprotein. The LDL cholesterol concentration is not measured directly, but is calculated by subtracting the HDL cholesterol and VLDL cholesterol values from the total cholesterol. Although this method is relatively rapid and inexpensive, there are several steps where error could be introduced. For example, accurate measurements of HDL depends on complete precipitation of apo-B containing lipoproteins. Traces of LDL in the supernatant can lead to overestimation of HDL cholesterol. Moreover, the multiple assumptions involved in the Friedewald formula make this method susceptible to error. The calculated LDL value has been demonstrated to be inaccurate in patients with elevated TG levels of greater than 200 mg./dl. In addition, this method does not provide a separate measure of IDL cholesterol or Lp(a) cholesterol. Instead, these values are included in the value calculated for LDL cholesterol.

Thus, it is desirable to provide a cholesterol testing system and method that overcomes the limitations of the conventional systems by measuring each cholesterol subclass directly from the raw data and it is to this end that the present invention is directed.

### SUMMARY OF THE INVENTION

A cholesterol testing system and method are described that include, in a preferred embodiment, a computer system that performs various operations and functions based on one or more piece of software code being executed by a processor of the computer system. The system and method includes an ultracentrifugation step which separates the different cholesterol types into density layers within the centrifugation tube. The ultracentrifugation step may be allowed to continue until an equilibrium point is reached, or the process may be sufficiently designed to allow non-equilibrium points to be repeatably reached.

The computer system is connected to a fluid handling system which pumps a prescribed volume of reagent and centrifuged analyte though a flow cell which is monitored by a multi-frequency spectrometer. The software executed by the computer system may perform the function of analyzing the data from the spectrometer of the testing system in order to generate a set of cholesterol values, such as a total cholesterol value, an HDL3 value, an HDL2 value, an LPA value, an LDL value, an IDL value and a VLDL value. The generation of the basis set of cholesterol values may be known as deconvolving of the cholesterol subclasses included in the data from the spectrometer. The software implements a numerical method for deconvolving the cholesterol subclasses from the data from the spectrometer and the fluid handling system. The preferred method may include the step of normalizing the gradient axis to the LDL peak rather than the more difficult to detect VLDL peak (although any suitable data landmark may be used). The method also includes the step of utilizing basis curves which can move along the gradient axis to better track slight process changes of the curve. The method may also remove additional process and measurement noise by allowing the basis curves to change in terms of shape and width. The method reduces the number of sub-curves utilized by the prior art which can remove numerical instabilities caused by overlapping or redundant curves. The software permits the testing lab to standardize final cholesterol results with external lab results (measured by an alternate method) and may also constrain final cholesterol values to positive values.

A system and method for determining cholesterol class values is described. The method and system may include a dynamic initialization process and then a deconvolution process in which the cholesterol class values are determined. In a preferred embodiment, the cholesterol class value determining method may use Weibull Equations as the basis curve function. A non-linear optimization process, such as the Levenberg Marquardt optimization modified to allow constraints on each parameter of amplitude, shape, location and width will allow full customization of the curve fit to each measured cholesterol profile. In another embodiment of the invention, the cholesterol class determining process may use a direct area measurement technique based on profile morphology.

### BRIEF DESCRIPTION OF THE DRAWINGS

FIG. 1 is diagram illustrating a controlled dispersion flow analysis system that may incorporate the cholesterol testing system in accordance with the invention;

FIG. 2 is a diagram illustrating a preferred embodiment of a dispersion flow analysis system that may incorporate the cholesterol testing system in accordance with the invention;

FIG. 3 is a diagram illustrating an example of a set of profiles generated by the controller dispersion flow analysis system shown in FIGS. 1 and 2;

FIGS. 4A and 4B are a flowchart of a method for cholesterol class determination in accordance with the invention;

FIG. 5 illustrates an example of four different Weibull curves;

FIG. 6 illustrates a parameter transform;

FIG. 7 is an example of the code used to generate the coefficients in accordance with the invention;

FIG. 8A is an exemplary profile and its time derivatives;

FIG. 8B is a diagram illustrating an example of a preprocessing process in accordance with the invention;

FIG. 9 illustrates an example of the feature locations for the profile shown in FIG. 8;

FIG. 10 is an example of a user interface showing the calculated cholesterol class values in accordance with the invention; and

FIG. 11 is an example of a profile with direct area measurement of the cholesterol class values in accordance with the invention.

### DETAILED DESCRIPTION OF A PREFERRED EMBODIMENT

The invention is particularly applicable to a software implemented cholesterol testing system and method and it is in this context that the invention will be described. It will be appreciated, however, that the system and method in accordance with the invention has greater utility since it may be implemented in other computer-based systems and may be used to determine other physiological parameters (e.g. protein concentrations, glucose concentrations, cellular types, etc.) using the same methodology as described below. Prior to describing the computer-implemented cholesterol testing system in accordance with the invention, a controlled dispersion flow analysis system that may incorporate the cholesterol testing system will be described.

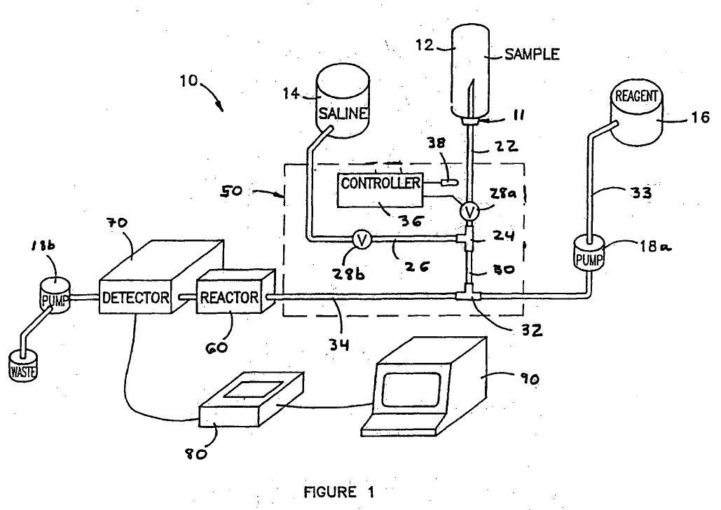

FIG. 1 illustrates a controlled dispersion flow analysis system 10. The controlled dispersion flow analysis system 10 includes a tube-piercing needle assembly 11 for piercing a sample tube containing a blood plasma sample which has been separated into individual lipoprotein classes by ultracentrifugation; a flow control sub-system 50 for mixing the sample with a reagent stream; a reaction unit 60 for reacting the sample with the reagent to produce a reaction signal indicative of the physiological parameter (cholesterol in the preferred embodiment) concentration in the sample; a detector unit 70 for monitoring the reaction signal and generating a physiological parameter profile, such as a cholesterol profile; and a computer 90 for collecting and analyzing the profile data. In a preferred embodiment on cholesterol determination, the profile may be an absorbance profile generated by a spectrometer and the computer 90 may analyze that profile to generate a plurality of cholesterol subclasses. In a preferred embodiment, the absorbance measured by the detector 70 is captured directly by the software being executed by the computer system. The computer 90 decomposes the absorbance data to make a quantitative determination of cholesterol in the individual lipoprotein classes and subclasses when the system is used to determine cholesterol. Further details of an example of a controlled dispersion flow analysis system that may incorporate the physiological parameter determination system and method in accordance with the invention is described in U.S. Pat. Nos. 5,633,168 and 5,284,773 which are incorporated herein by reference.

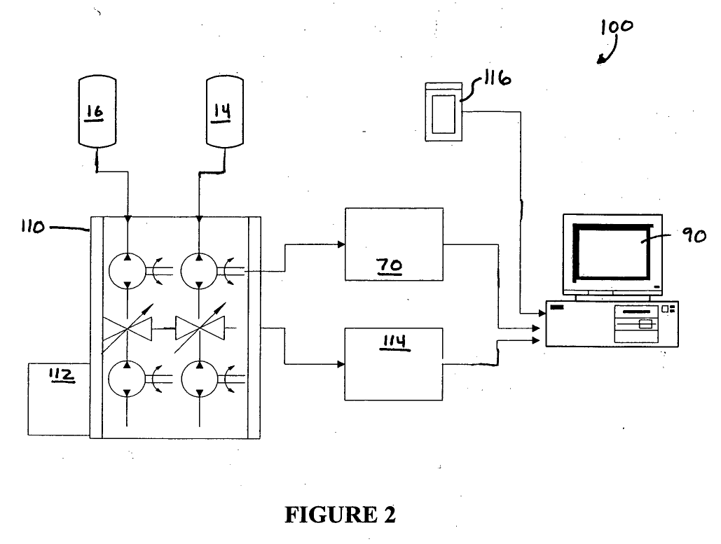

FIG. 2 is a diagram illustrating a preferred embodiment of a dispersion flow analysis system 100 that may incorporate the cholesterol determination system in accordance with the invention. As shown, the system 100 may include the reagent container 16 and the saline container 14 and the computer 90 shown in FIG. 1. The system shown in FIG. 2 operates in a substantially similar manner to the system shown in FIG. 1. The system 100 also include a multiple channel vertical auto profile (MCVAP) unit 110 and a programmable logic controller 112 that controls the mechanical operation of the MCVAP unit. In accordance with the preferred embodiment of the system 100, the MCVAP is capable of testing multiple samples (four samples in the embodiment shown in FIG. 2). Each sample is then fed through the detector 70, that may preferably be a spectrometer, that may be preferably connected to the computer 90. The detector transmits the data measured from the sample (an absorption profile of the sample in the cholesterol determination embodiment described above) to the computer 90. The system 100 may also include a digital input/output board 114 and a scanner 116, such as a barcode scanner, that are connected to the computer 90 preferably using a PCI bus and a keyboard port, respectively. The scanner may be used to scan a barcode of a sample vial so that each sample can be identified within the testing system.

The computer 90, in a preferred embodiment, may be a typical computer system with sufficient processing power and memory to execute the one or more pieces of software that are part of the cholesterol testing system. For example, the computer 90 may be a personal computer with a 2.0 GHz processor, 40 GB hard drive, 256 MB RAM minimum and the other components shown in FIG. 2. In a preferred embodiment, the computer 90 may use the Windows 2000 or Windows XP operating system and the one or more pieces of software of the testing system may be written in Visual Basic and C# using the NET platform and development environment.

The software of the testing system may perform various functions that includes monitoring the detector output to provide a measured cholesterol value for each of the supported subclasses for each sample. The measured cholesterol value for each of the supported subclasses may then be stored in a storage unit (not shown) connected to the computer 90, such as a database, table, etc. so that the values are available for subsequent queries and results distribution as determined by the customer. Then, after the samples are run and the results calculated, the user will be presented with the results of the calculation. Multiple levels of user-determined review and approval options will be available. Technician input may be required at various points in the process (segmentation, control verification) but will not be required under normal circumstances. All of the results and calculation parameters will be stored in the storage unit. The system will have access menus/GUI's for reviewing and setting process and quality control limits utilized by the system wherein the calibration and control features will be integrated, and can be run at the discretion of the user. The results that may be stored in the storage unit may include, for each sample: a specimen ID, Gender, dilution factor, control status; total Cholesterol, all cholesterol subclasses; the 3 major cholesterol classes (HDL, LDL and VHDL); the LDL pattern; the software version; the instrument ID; and a run date/time. A printout of the results can be generated at anytime after the results are complete.

The software of the testing system may include five components including 1) POSID software, 2) Data Collection Software, 3) SAVAP software, 4) CALIB software and 5) the SAVAP database which is data structure into which the results and other data of the testing systems are stored in the storage unit. The POSID software allows the User to scan the Sample Carrier barcode, then individually scan the Sample ID barcode. Working in conjunction with the POSID hardware fixture (optical sensor pad), the specimens are identified and their location is recognized. The POSID Software application will display a GUI user interface, enabling user input which includes the identification of control samples, rotor assignment, and dilution factor assignment. The GUI interface allows the User to correct errors made during the bar code scanning process. The GUI interface also allows to manual data entry in lieu of bar code scanning. The POSID software application writes the specimen location and input data to the central SAVAP Database (SAVAP-DB).

The Data Collection software is responsible for monitoring and recording data from four separate spectrophotometric channels. The user activates the Data Collection process through the GUI interface. The GUI interface allows the user to perform a check of the spectrometer channel relative intensities prior to commencing Data Collection. At periodic intervals, the Data Collection subsystem calculates and Absorbance Value from the captured raw spectrums. An absorbance value is saved at each time step for each spectrometer channel. The raw spectrum data is not saved. The raw absorbance and valve data is stored in memory until the completion of the batch when it is then moved from memory to the SAVAP Database.

The CALIB software is utilized by the user when lab personnel determine that machine calibration is required. The CALIB software is used to calculate new calibration factors. The CALIB system is a Windows application that operates independently from the SAVAP application; but utilizes the data available in the SAVAP_DB. It consists of a top level Windows GUI that determines the data to use in the calculation. A calculation engine performs all required calculations, including outlier statistics and reports the Calibration Factor for all lanes of the machine.

The SAVAP software is responsible for preprocessing, segmenting, calculating, approving all cholesterol results. The application also contains several maintenance and set-up operations. The SAVAP software has a GUI interface termed “Dashboard” that enables control of the Data Collection, System Configuration, Control Maintenance, Search/Report functions as well as approval of the batch by User personnel. After the samples are processed and the results calculated, the user is presented with the results of the calculation and a software “accept/reject” decision. All results of the calculation and parameters will be stored in the SAVAP_DB. The software will have a Manual Segmentation feature that allows the user to override the autosegmentation function and segment the lane of profile data into individual specimen profiles manually. Now, the SAVAP software and its deconvolution process in accordance with the invention will be described in more detail.

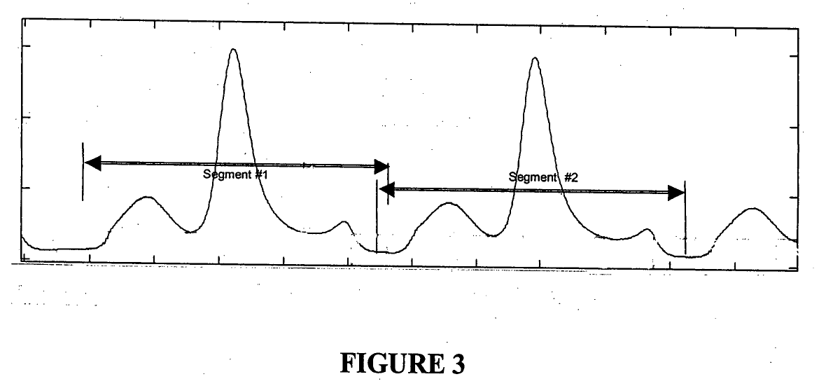

FIG. 3 is a diagram illustrating an example of a set of profiles generated by the controller dispersion flow analysis system shown in FIGS. 1 and 2. As is well known, during a vertical auto profile process involving centrifugation described above, a sample is centrifuged so that the different density cholesterol types form vertical strata in the sample vial so that the types/classes are separated from each other. Then, as the sample vial is punctured, the different strata flow out of the vial and passes the detector (a spectrometer in the preferred embodiment) that generates an absorption profile such as those shown in FIG. 3. However, the separation is not complete and the classes are based on ranges of density values. Therefore, rather than giving a sharp peak (as in IR spectroscopy), a distribution or curve for each type/class of cholesterol is produced and these curves overlap and when summed, produce the observed profile shown in FIG. 3. Thus, in order to generate a value for each different type/class of cholesterol from the profile, it is necessary to determine the combination of curves for all of the classes of cholesterol that fit the profile. The curve for each class of cholesterol will permit a value for each class of cholesterol to be accurately determined. Now, a method for cholesterol class determination in accordance with the invention will be described in more detail.

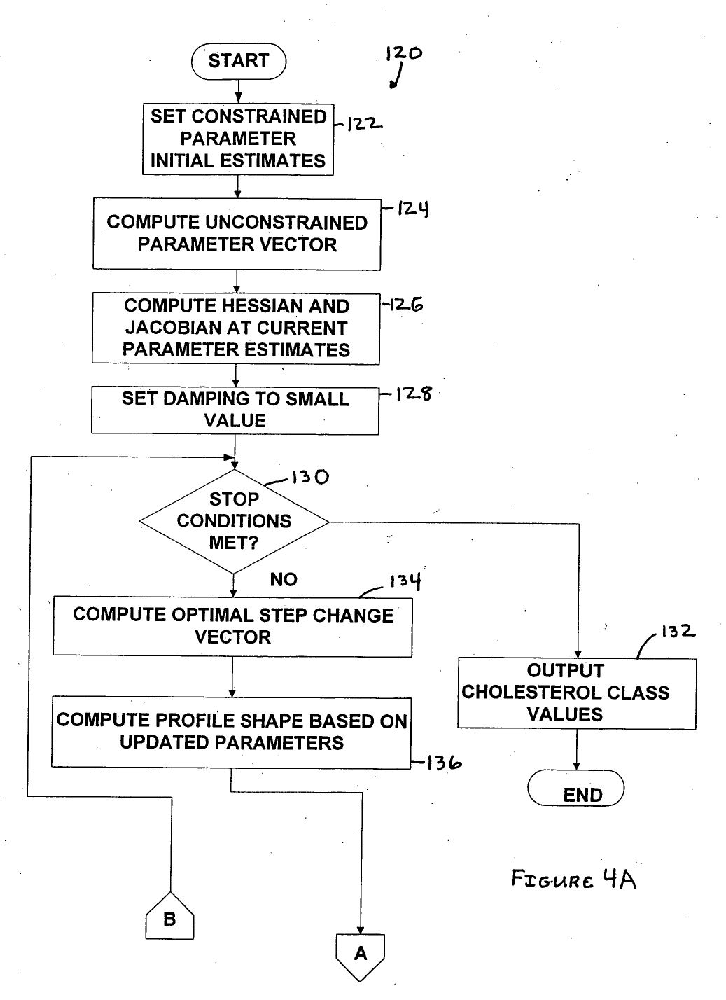

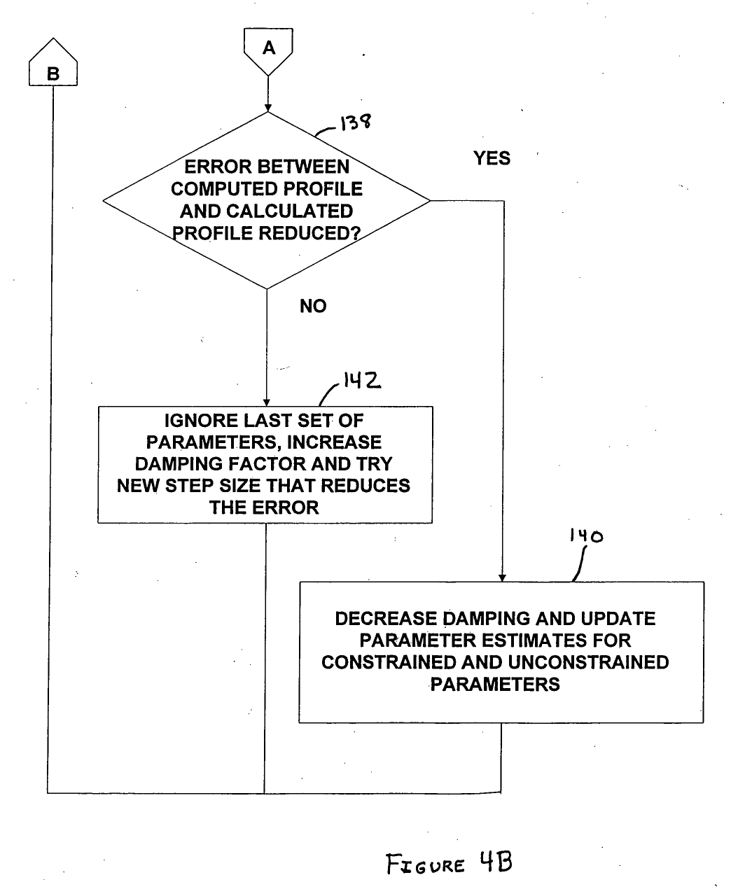

FIGS. 4A and 4B are a flowchart of a method 120 for cholesterol class determination in accordance with the invention. In a preferred embodiment of the invention, a Weibull Equation may be used to define the basic sub-curve shape. In particular, the Weibull Equation is a fairly well known commodity in statistical and reliability calculations that may be used to generate the combination of cholesterol class curves that fit the profile generated by the spectrometer. In the preferred embodiment, the Weibull Equation may be used because it requires only four parameters, assumes a wide continuum of curve shapes and sizes, the area under any unit amplitude curve is always =1.00 and the equation is analytically differentiable on all four parameters.

The standard Weibull equation has the following form in which all of the curves are defined at any point X by a single equation:

$$\frac{β}{{α}^{β}} \cdot {( X - μ )}^{β - 1} \cdot {e}^{- {( \frac{X - μ}{α} )}^{β}} \qquad \text{Equation 1.1}$$

where X is a particular point on the curve;

α controls the curve width;

β control the curve shape; and

μ controls the curve location.

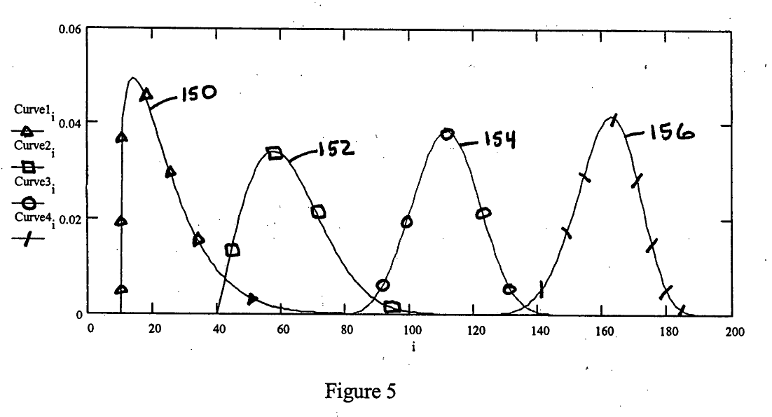

FIG. 5 illustrates an example of four different Weibull curves. A first curve 150 is generated when the α=15; β=1.25 and μ=10. A second curve 152 is generated when the α=25; β=2 and μ=40. A third curve 154 is generated when the α=35, β=3.5 and μ=80. A fourth curve 156 is generated when the α=45; β=5 and μ=120. Thus, by simply varying the parameter values, a wide variety of curve shapes and sizes are possible. This is advantageous because it allows the deconvolution process to account for laboratory and process variations which may alter the expected shape, location and/or size of the basis curve.

In order to use the Weibull Equation as the basis for determining the cholesterol subcurves, it is desirable to constrain the parameters to physiological and hydrodynamically feasible ranges. In particular, the Amplitude, which is directly related to the final cholesterol value, must be greater than or equal to zero. In addition, the location parameter, μ, must be limited to a feasible range along the gradient axis relative to the initial starting position. For example, LDL curves should float between the starting point dictated by the feature detection process of this invention within ±15 on the scale (where, in a preferred embodiment, the LDL peak is scaled to 100.0), rather than having free rein over the entire range. Based on flow dynamics of cholesterol pumping through a tube, it is not expected that any subcurve will have a purely exponential form, so β>1.0 could also be a valid constraint.

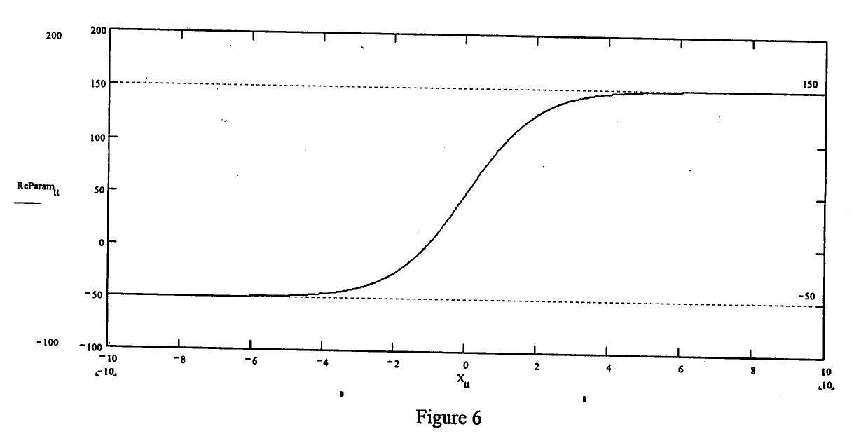

While there are a number of linear optimization routines that can utilize parameter constraints, there are only a limited number of non-linear algorithms that can handle constraints. These algorithms make use of active sets and high levels of iteration to converge to a feasible solution. An alternate approach is to use a non-linear optimization routine for unconstrained parameters but use transformed parameters. The original parameters are transformed to provide a constrained output and the unconstrained results are back transformed after a solution is found. To limit a parameter to the range [1α. . . uα], the preferred transformation (and inverse transform) used in the cholesterol determining method in accordance with the invention are:

$$q α = 1 α + Δ α \cdot ( \frac{{e}^{α}}{1 + {e}^{α}} ) \\ α = ln [ \frac{- ( q α - 1 α )}{q α - 1 α - u α} ] \qquad \text{Equations 1.20 and 1.21}$$

where Δα=uα−1α is the desired range above the lower limit. The original parameter, α is now constrained and the transformed parameter, qα, can run over all real numbers (ie. unconstrained). The effects of this transformation are readily plotted. For example, to limit a parameter to take only values in the interval [−50, 150], the transform would be as shown in FIG. 6. An unconstrained optimization could be run on the transformed variable X which is defined over all real numbers. Obviously unconstrained values below −10 map back to original values of −50; unconstrained values greater than +10 will map back to the constrained value of 150.

Now, the constrained derivatives may be calculated. In particular, using a constrained transformation for each of the four Weibull parameters, the Weibull equation becomes

$$qA \cdot \frac{q β}{q {α}^{q β}} \cdot {( X - q μ )}^{q β - 1} \cdot {e}^{- {( \frac{X - q μ}{q α} )}^{q β}} \qquad \text{Equation 1.3}$$

where each parameter is transformed with the constrained Equation 1.3. Fully expanded, the transformed Weibull definition becomes:

$$[ 1 A + Δ A \cdot ( \frac{{e}^{A}}{1 + {e}^{A}} ) ] \cdot \frac{1 β + Δ β \cdot ( \frac{{e}^{β}}{1 + {e}^{β}} )}{{[ 1 α + Δα \cdot ( \frac{{e}^{α}}{1 + {e}^{α}} ) ]}^{1 β + Δβ \cdot ( \frac{{e}^{β}}{1 + {e}^{β}} )}} \cdot {[ x - [ 1 μ + Δμ \cdot ( \frac{{e}^{μ}}{1 + {e}^{μ}} ) ] ]}^{1 β + Δβ \cdot ( \frac{{e}^{β}}{1 + {e}^{β}} ) - 1} \cdot {e}^{- {[ \frac{X - [ 1 μ + Δμ \cdot ( \frac{{e}^{μ}}{1 + {e}^{μ}} ) ]}{1 α + Δα \cdot ( \frac{{e}^{α}}{1 + {e}^{α}} )} ]}^{1 β + Δβ \cdot ( \frac{{e}^{β}}{1 + {e}^{β}} )}} \qquad \text{Equation 1.4}$$

Rewriting Equation 1.3 as StdWeibull(qA,qα,qβ,qμ) and defining

$$EAB ( λ ) = \frac{{e}^{λ}}{1 + {e}^{λ}}$$

$$Xmab = {( \frac{X - μ}{α} )}^{β}$$

The partial derivatives of Equation 1.4 with respect to each parameter can then be found in closed form which are required by many optimization routines in order to speed convergence.

The derivative with respect to amplitude (A) is:

$$\frac{StdWeibull ( qA , q α , a β , q μ )}{Amp} \cdot {Δ}_{A} \cdot ( EAB ( A ) - {EAB ( A )}^{2} )$$

The derivative with respect to alpha is:

$$StdWeibull ( qA , q α , a β , q μ ) \cdot {Δ}_{α} \cdot [ \frac{q β}{q α} \cdot ( EAB ( α ) - {EAB ( α )}^{2} ) ] \cdot [ - 1 + {( \frac{X - q μ}{q α} )}^{q β} ]$$

The derivative with respect to Beta is:

$$StdWeibull ( qA , q α , a β , q μ ) \cdot {Δ}_{β} \cdot ( EAB ( β ) - {EAB ( β )}^{2} ) ( \frac{1}{β} - ln ( q α ) + ln ( X - q μ ) - Xmab \cdot ln ( \frac{X - q μ}{q α} ) )$$

The derivative with respect to Mu is:

$$StdWeibull ( qA , q α , a β , q μ ) \cdot \frac{1}{X - q μ} \cdot {Δ}_{μ} \cdot ( EAB ( μ ) - {EAB ( μ )}^{2} ) ( 1 - q β + q β \cdot Xmab )$$

Note that all derivatives are functions of both the original, constrained parameters and the transformed, unconstrained parameters. Therefore, the algorithm must convert back and forth at each iteration of the optimization routine by the use of Equations 1.20 and 1.21. However, one skilled in the art will recognize that all calculations could be performed in the unconstrained domain with substantially more complicated expressions. Upon convergence, the unconstrained parameter solution vector could be transformed back to the desired constrained values.

Returning to FIGS. 4A and 4B, the method begins when the parameters are dynamically initialized. In particular, the method allows the subcurves to float in location during the iteration process, but also initializes the location based on morphological features of the measured profile. This dynamic initialization speeds convergence and ensures that the subcurves remain in the correct order. The dynamic initialization also permits the method to use a process that does not depend on any subcurves at all as described below in more detail. As shown, in step 122, the constrained parameter initial estimates based on profile shape are set. As described above, certain parameters are constrained to physiological and hydrodynamically feasible ranges wherein 1) the amplitude must be greater than or equal to zero; 2) the location parameter, μ, must be limited to a feasible range; and 3) due to flow dynamics of cholesterol pumping through a tube, the curves should not have a purely exponential form so that the value of beta is greater than 1.0.

The initial locations of the curves are determined by the feature vector which is described below. Once the initial location for each subcurve is determined, the feasible range is set relative to this initial location. In a preferred embodiment using a preferred set of process settings (including flow rate, reagent ratio, tubing lengths and tubing diameters, the values presented in Table 2 below show the constrained range for the location parameter, μ.

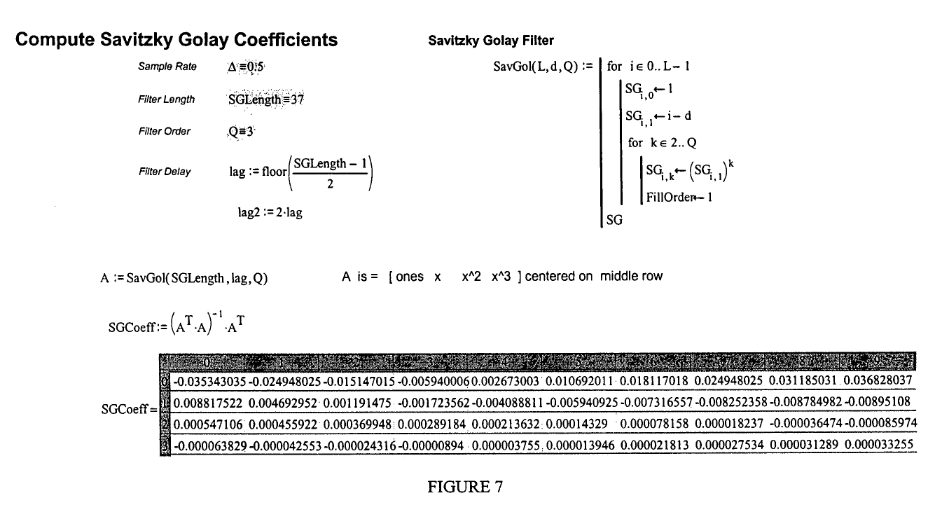

In step 124, the unconstrained parameter vector is computed. The feature vector is based on the measured profile, and its first and second derivatives with respect to time. Note that these derivatives are based on the entire profile and are independent of the form of the subcurve equation. The derivatives are calculated, in a preferred embodiment, using the well known Savitzky Golay filter designed with a cubic interpolation (Order 3) and 37 profile points (Length=37). While not required, the preferred embodiment of the method uses the known sample rate to ensure that the first and second derivatives are calculated in the proper units (Absorbance/sec, and Absorbance/sec², respectively). The Savitzky Golay filter coefficients could be calculated offline and hard coded into the source code or calculated at runtime. Derivatives are calculated on a low pass filtered version of the measured profile. Calculations are performed as a direct implementation of the mathematical convolution operation (not related to subcurve decomposition). Filter lags at the start and end of the derivative vectors are truncated to the correct length. FIG. 7 illustrates an example of the code that may be used to generate these coefficients and the Savitzky Golay filter used in the preferred embodiment of the invention.

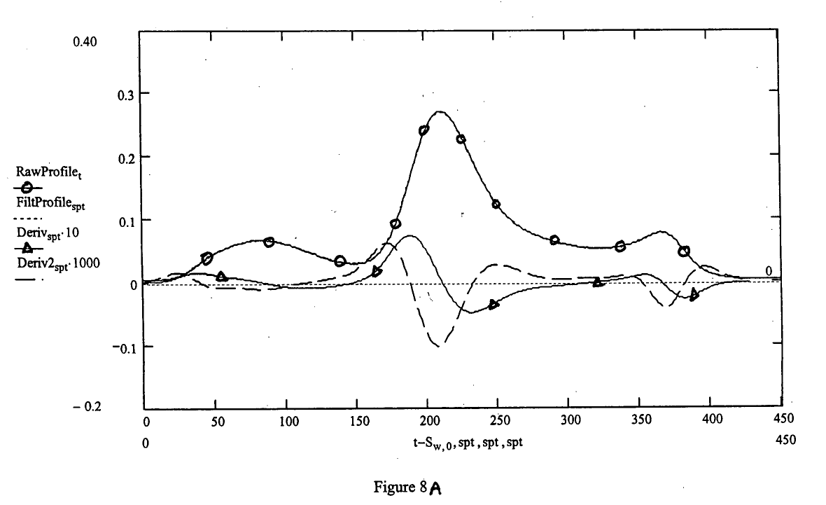

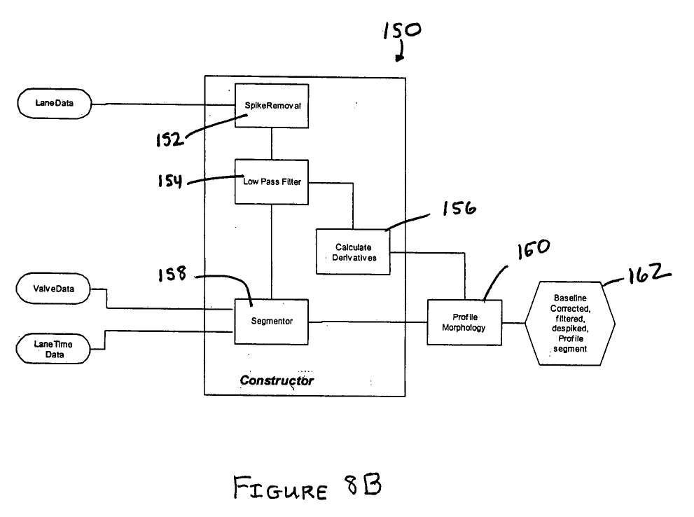

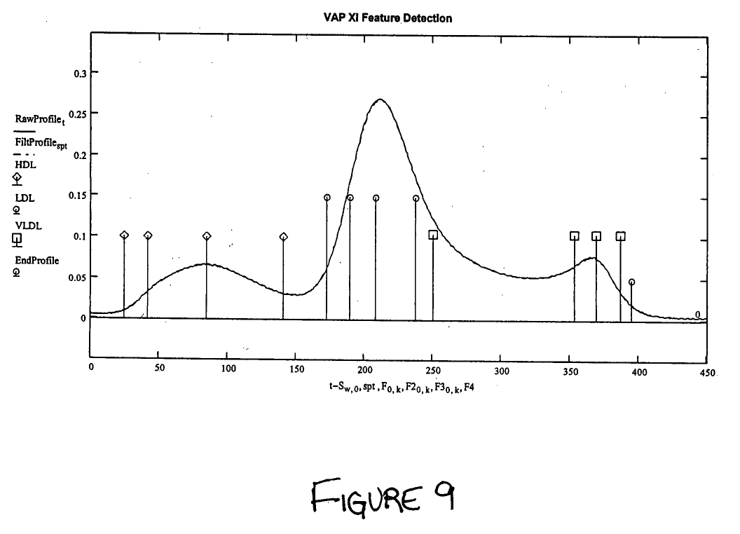

A sample profile and its time derivatives are shown in FIG. 8A. In this figure, the derivative values have been scaled for plotting purposes to fit on the same scale as the absorbance profiles. A feature search process tries to find four features for each of the HDL, LDL, and VLDL peaks for a total of 12 features. FIG. 8B is a diagram illustrating an example of a preprocessing process 150 in accordance with the invention during which the feature search process may occur. The preprocessing process may operate on raw profile data and may include a spike removal step 152 during which spikes (wherein the spikes may be caused by bubbles in the samples or other debris) are removed from the raw data and a low pass filter step 154 that removes high frequency noise that is not associated with the cholesterol measurement. The output of the spike removal and low pass filter is fed into a derivative calculator 156 and a segmentor 158 wherein the derivative calculator calculates the time derivatives of the profile, an example of which is shown in FIG. 8A. The segmentor 158 may calculate the features of each profile such as that shown in FIG. 9. In a profile morphology step 160, the output of the time derivatives and the segmentor are combined to generate the profile and an array of specific morphological features measured in each profile. The four Features found for each of the three major peaks of the profile are:

TABLE 1

| Name | Description |
|---|---|
| MaxD2 | Maximum second derivative. Highest curvature |
| MaxPosSlope | Maximum positive slope on the leading edge of the Peak. |
| Peak | First negative value of the first derivative after a |
| | minimum second derivative value |
| MaxNegSlope | Maximum(most) negative slope. |

The 13^{th} feature is the estimate of the end profile point. In addition to these parameters that are determined based on the profile, the location parameter (as discussed above) for each of the predetermined number of subcurves (there are 11 subcurves in a preferred embodiment of the method) is initialized relative to the values contained in the feature array. For example, the profile depicted in FIG. 9 would generate a Feature vector of:

FeatureIndex=[25 47 83 143 170 182 205 238 252 355 370 385 395]

In particular, the subcurve starting locations are interpolated between any two features using empirically determined interpolation ratios which are hard coded and constant for all profiles but have been shown to get each subcurve ‘in the neighborhood’ for a large number of tested profiles. Since the location parameters are part of the optimization routine, the iteration calculation is responsible for fine tuning the location in conjunction with the other three parameters defining a subcurve. An example of the interpolation ratios in the preferred embodiment of the method (for an algorithm using 12 subcurves) is:

TABLE 2

| Sub- | | | | |
|---|---|---|---|---|
| curve | Name | Initial Location | Lower | Upper |
| 0 | HDL3b | F[0] | 0.0 | IL + 9 |
| 1 | HDL3a | 0.5 * F[0] + 0.5 * F[1] | IL − 15 | IL + 8 |
| 2 | HDL2 | 0.25 * F [1] + 0.75 * F [2] | IL − 12 | IL + 8 |
| 3 | LpA | 0.15 * F [2] + 0.85 * F [3] | IL − 3 | IL + 6 |
| 4 | LDL4 | 0.25 * F[3] + 0.75 * F[4] | IL − 8 | IL + 8 |
| 5 | LDL3 | 0.75 * F[3] + 0.25 * F[4] | IL − 8 | IL + 8 |
| 6 | LDL2 | 0.15 * F [4] + 0.85 * F[5] | IL − 8 | IL + 8 |
| 7 | LDL1 | 0.75 * F[6] + 0.25 * F[7] | IL − 8 | IL + 8 |
| 8 | IDL | 0.75 * F[8] + 0.25 * F[9] | IL − 6 | IL + 6 |
| 9 | VLDL 3 | 0.5 * F[8] + 0.5 * F[9] | IL − 8 | IL + 12 |
| 10 | VLDL2 | 0.35 * F[8] + 0.65 * F[9] | IL − 13 | IL + 18 |
| 11 | VLDL1 | 0.25 * F[9] + 0.75 * F [10] | IL − 25 | 0.75 * F + |
| | | | | 0.25 * F[11] |

In Table 2, F[i] represents the ith element in the Feature array. IL is the initial location defined by the third column. The lower range allowed for curve 0 is an absolute value of 0.0. All other ranges are relative to the initial location with the exception of the upper range for the last curve which is defined relative to the last two feature values.

The interpolation constants (the third column in Table 2) and the range constants (the fourth and fifth columns in Table 2) are dependent on the flow and chemical conditions of the hydrodynamic process. The values in Table 2 are provided here as an exemplary set of values that has been shown to work with the preferred process embodiment. However, it should be obvious that these constants are likely to change (and those changes are within the scope of the invention since the user may regularly adjust the parameters in response to changing lab and process conditions) as the processes evolve and unknown variations occur in the normal laboratory environment.

In a similar manner, the values and ranges of the Alpha parameter can be initialized based on the values in the Feature array. This is advantageous since any change in scaling will be compensated by shrinking or enlarging the initial size of each subcurve. Table 3 shows an exemplary set (for an algorithm using 12 subcurves) of initial values and ranges for initializing the Alpha parameter.

TABLE 3

| Subcurve | Name | Initial Alpha | Lower | Upper |
|---|---|---|---|---|
| 0 | HDL3b | 1.4 * (F[1] − F[0]) | 0.5 * IA | 1.5 * IA |
| 1 | HDL3a | 1.25 * (F[1] − F[0]) | 0.5 * IA | 1.5 * IA |
| 2 | HDL2 | 1.25 * (F[3] − F[2]) | 0.5 * IA | 1.5 * IA |
| 3 | LpA | 1.00 * (F[5] − F[4]) | 0.5 * IA | 1.5 * IA |
| 4 | LDL4 | 1.00 * (F[6] − F[5]) | 0.5 * IA | 1.5 * IA |
| 5 | LDL3 | 1.00 * (F[6] − F[5]) | 0.5 * IA | 1.5 * IA |
| 6 | LDL2 | 1.00 * (F[6] − F[5]) | 0.5 * IA | 1.5 * IA |
| 7 | LDL1 | 1.00 * (F[6] − F[5]) | 0.5 * IA | 1.5 * IA |
| 8 | IDL | 1.00 * (F[6] − F[5]) | 0.5 * IA | 1.5 * IA |
| 9 | VLDL3 | 1.00 * (F[8] − F[6]) | 0.5 * IA | 1.5 * IA |
| 10 | VLDL2 | 0.61 * (F[11] − F[9]) | 0.5 * IA | 1.5 * IA |
| 11 | VLDL1 | 1.00 * (F[11] − F[9]) | 0.5 * IA | 1.5 * IA |

In Table 3, IA is the initial alpha in column 3 of Table 3. As with the location parameters, the constants and Feature index used in Table 3 are exemplary and can be adjusted by laboratory personnel to account for normal laboratory variations or intentional process changes such as increasing the flow rates to increase throughput.

FIG. 9 illustrates an example of the feature locations for the profile shown in FIG. 8. In most cases, profile look relatively similar and convergence can be sped up if the initial Beta values are started in the general vicinity of the final, average parameters. The initial values and allowed ranges for the Beta parameters in the preferred embodiment (for an algorithm using 12 subcurves) are detailed in Table 4.

TABLE 4

| Subcurve | Name | Initial Beta | Lower | Upper |
|---|---|---|---|---|
| 0 | HDL3b | 3.5 | 2.1 | 4.5 |
| 1 | HDL3a | 3.5 | 2.1 | 4.0 |
| 2 | HDL2 | 3.5 | 2.9 | 4.0 |
| 3 | LpA | 3.4 | 2.5 | 4.0 |
| 4 | LDL4 | 3.2 | 2.2 | 5.0 |
| 5 | LDL3 | 3.2 | 2.2 | 5.0 |
| 6 | LDL2 | 3.2 | 2.2 | 5.0 |
| 7 | LDL1 | 3.2 | 2.2 | 5.0 |
| 8 | IDL | 1.9 | 2.5 | 4.5 |
| 9 | VLDL3 | 3.2 | 2.5 | 4.5 |
| 10 | VLDL2 | 2.7 | 1.9 | 4.5 |
| 11 | VLDL1 | 2.7 | 1.9 | 4.5 |

The values in Table 4 were determined empirically based on a data set collected in accordance with the protocol required for FDA submission. (See National Reference System for Cholesterol CRMLN Total Cholesterol Certification Protocol for Manufacturers, October 2004) In particular, two hundred samples were collected from healthy volunteers and tested under the present invention and process settings and compared to values supplied by a national reference laboratory using an independent method (enzymatic quantification). The initial values and allowed ranges were empirically modified to minimize the difference in results between the national reference laboratory and the values measured using the present invention.

In a preferred embodiment, it was found that converging to the same local minimum was enhanced by starting all Amplitude parameters at 10.0. This is a very large value for the subcurves used which will generally converge to values less than one. However, starting the Amplitude far from the expected solution, allows the Alpha, Beta, and Location parameters to take larger steps and converge to the same relative location each time. This provides more repeatable cholesterol values that are not as dependent on how close the initial guess is to the final solution.

As one skilled in the art will recognize, it is also possible to weight the parameters such that the optimization routine will more preferentially adjust one parameter (such as Amplitude) over the other-three parameters. This can be most readily accomplished by scaling the parameters in the Weibull equation such that the derivatives will dominate the search process. The scaling factor can then be reversed after the optimization is concluded.

Once the parameters have been initialized, a non-linear optimization process is carried out in step 126 using the Weibull Equations and additional functions that may be required by the optimization routine used. In a preferred embodiment, the two additional functions may be the Jacobian and Hessian vectors for the Weibull Equations and the non-linear optimization may be the Levenberg Marquardt optimization that is well known in digital signal processing literature. The Levenberg Marquardt optimization routines are often used in non-linear least squares fitting problems because both the Jacobian and the Hessian matrix for the fitting errors are dependent only on the fitting equation and its partial derivatives.

The Levenberg Marquardt optimization method depends on converting a non-linear, possible constrained, optimization problem into a linear, unconstrained problem. The method begins by defining a suitable function that measures the degree of fit between the measured data and the calculated, fitted data. The preferred function is the least squares measure that will now be described in more detail. The error function is:

Error Function

$$X ( i ) = {\sum }_{i = 1}^{NumPts} {( {Meas}_{i} - {Fitted}_{i} )}^{2}$$

wherein Measᵢ is the measured curve value at point i and Fittedᵢ is the fitted curve value at point i and the NumPts is the number of points over which the error function is calculated. The values which will provide the best fit, in this least squares sense, are found where the derivative of this function is zero. This can be found as (ignoring constants):

Derivative of Error Function

$$\frac{δ X}{δ {p}_{k}} = {\sum }_{i = 1}^{NumPts} [ ( {Meas}_{i} - {Fitted}_{i} ) \cdot \frac{δ W}{δ {p}_{k}} ] = 0$$

where

$$\frac{δ W}{{δ}_{{p}_{k}}}$$

is the partial derivative of the Fitting function for Parameter k.
and

$$\frac{δ X}{{δ}_{{p}_{k}}}$$

is the partial derivative of the Error Function for parameter k

The Error function Hessian matrix (shown as Equation 1.52) is made up of the second derivatives (calculated as discussed above) and can be considered the curvature matrix for least squares solutions. Taking another derivative provides the Hessian of the error function which is:

Hessian of Error Function
Taking another derivative, gives the Hessian of the Error Function

$$\frac{{δ}^{2} \cdot X}{δ {p}_{j} \cdot δ {p}_{k}} = {\sum }_{i = 1}^{NumPts} \cdot [ ( \frac{δ W}{δ {p}_{j}} \cdot \frac{δ W}{δ {p}_{k}} ) - ( {Meas}_{i} - {Fitted}_{i} ) \cdot \frac{{δ}^{2} \cdot W}{δ {p}_{j} \cdot δ {p}_{k}} ] \qquad$$

where

$$\frac{{δ}^{2} \cdot W}{{δ}_{{p}_{j}} \cdot {δ}_{{p}_{k}}}$$

is the second partial derivative of the Fitting function to Parameters j and k

Thus both the Jacobian and the Hessian matrix of the least squares solution are fully defined for Weibull subcurves by the partial differential equations discussed above.

The above Equation 1.5 is often simplified by ignoring the second derivative terms. Rewriting the partial derivatives of the error function in terms of the two matrices and converting to matrix notation results in:

Jacobian and Hessian of the Error Function
For a four parameter curve fit, the Jacobian and Hessian matrices can be determined analytically. They are shown in symbolic form here.

$$\begin{matrix}Jacobian = ( \begin{matrix}\frac{δ W}{δ A} \\ \frac{δ W}{δα} \\ \frac{δ W}{δ β} \\ \frac{δ W}{δ μ}\end{matrix} ) & \text{EQUATION 1.61} \\ Hessian = [ \begin{matrix}{( \frac{δ W}{δ A} )}^{2} & \frac{{δ}^{2} W}{δ A δ α} & \frac{{δ}^{2} W}{δ A δβ} & \frac{{δ}^{2} W}{δ A δμ} \\ \frac{{δ}^{2} W}{δ A δ α} & {( \frac{δ W}{δα} )}^{2} & \frac{{δ}^{2} W}{δαδ β} & \frac{{δ}^{2} W}{δαδ μ} \\ \frac{{δ}^{2} W}{δ A δ β} & \frac{{δ}^{2} W}{δαδ β} & {( \frac{δ W}{δβ} )}^{2} & \frac{{δ}^{2} W}{δβδμ} \\ \frac{{δ}^{2} W}{δ A δ μ} & \frac{{δ}^{2} W}{δαδμ} & \frac{{δ}^{2} W}{δβδμ} & {( \frac{δ W}{δμ} )}^{2}\end{matrix} ] & \text{EQUATION 1.62}\end{matrix}$$

In the case of cholesterol determining method, the partial derivatives of the constrained, four parameter, Weibull equation make up the Error function Jacobian vector shown as Equation 1.61. It can be seen from Equations 1.61 and 1.62 that a final simplifying notation can be made such that the partial derivative of the Error function is:

$$\frac{δ X}{δ {p}_{k}} = {J}^{T} \cdot e$$

where J=Jacobian of the Error Function
and e e=Meas−Fitted is the Error Vector

And the second partial derivative of the Error function is:

$$\frac{{δ}^{2} \cdot X}{δ {p}_{j} \cdot δ {p}_{k}} = ( \frac{δ W}{δ {p}_{j}} \cdot \frac{δ W}{δ {p}_{k}} ) = {J}^{T} \cdot J$$

If the least squares assumptions are valid, and the second derivatives can truly be ignored relative to the Jacobian terms, the optimal solution can be found in a single iteration by solving for the inverse of the Hessian matrix and multiplying by the gradient (Jacobian) vector so that:

ParameterChange=−(Hessian⁻¹*Jacobian)

gives a column vector of changes to make to each parameter to find the local optimizers.

However, if in the local region the least squares assumptions are not particularly appropriate, then the best solution is to simply take a step in the steepest descent direction. The Levenrberg Marquardt algorithm introduces a damping factor which can smoothly vary between these two approaches. Thus, in step 128, the method sets this damping factor to a small value. In a preferred embodiment, an initial damping value (“Initial MU”) is set to 0.51. As set forth below in more detail, the value is doubled each time an iteration creates an improvement in the curve fit that allows the next iteration to take a “bigger step” down the gradient surface. If an iteration step creates a worse curve fit as described below, the damping factor is cut in half and another iteration is tried with a smaller step.

Using the damping factor, the Levenberg Marquardt optimization is performed using iterations wherein the constrained parameter estimates are kept in synch with the unconstrained parameter estimates. Normally, the fitted parameters would be plugged into the subcurve equation and integrated to find the area under each subcurve. This area is directly proportional to the cholesterol level in that subclass. If the curve fit to the measured profile data is sufficiently good, the areas will sum to the area under the measured profile curve. The Weibull subcurves will always have an integrated area=1.00 for Amplitude=1.0. Therefore, for this method, the subcurve area is simply the Amplitude parameter, and no further integrations are required. Now, the Levenberg Marquardt optimization in accordance with the preferred embodiment of the invention will be described in more detail.

As described above, the initial damping factor (Initial MU) of 0.51 is used and the damping factor is dynamically adjusted based on the outcome of the last iteration. The initial Hessian of the error function is calculated using the equations set forth above. In order to adjust the damping factor, the maximum value along the diagonal of the Hessian is used to scale the damping factor.

For each iteration:

MaxHessian=FindMaxDiagonal(HessianMatrix)

NextMU=LastMU*MaxHessian

The Hessian matrix diagonal is then augmented by adding the value of the NextMU to each element in the diagonal so that:

Hessian_{j,j}=Hessian_{j,j}+NextMU

The off diagonal elements are not altered. This has the desired effect of blending the algorithm smoothly between the steepest descent solution and the InvertHessian solution. As NextMU gets large, the Hessian becomes diagonally dominant and the optimum solution approaches the Steepest Descent method. As NextMU gets small, the solution is deemed relatively close and the direct Inverted Hessian solution can be used.

The next estimate for the Parameter vector is derived from the Levenberg Marquart augmented equation that is:

Hessian·ΔP=J^{T}·e

This is a linear equation in the ΔP and can be solved by ordinary least squares methods. Once the ΔP are found, the parameter estimates are updated and the Fitting function, Error Jacobian and Error Hessian are recalculated as:

Pₖ=Pₖ+ΔPₖ for each of the k parameters

Using the error function which is:

$$X ( i ) = {\sum }_{i = 1}^{NumPts} {( {Meas}_{i} - {Fitted}_{i} )}^{2}$$

the curve fit is determined to be better or worse. If the above error function is smaller, the it is assumed that the fitting is headed in the correct direction so that the Parameters values are kept and the damping factor is reduced wherein:

$$LastMU = \frac{LastMU}{AdjustMU}$$

Thus, in step 130, the software executing on the computer system determines if the stop conditions for the process have been met and the iterations are completed. In a preferred embodiment, the stop conditions occur when 1) a predetermined maximum allowed iterations has occurred; 2) the target level for the curve fit error has been reached (i.e. the fit is good enough); or 3) the parameter step changes have stopped changing (this may or may not be a good fit.) If the curve fit gets worse, then the error function is bigger and the fitting is worse and the method resets the last Parameter vector and increases the damping factor to reduce the step size of the next iteration. So,

LastMU=LastMU*AdjustMU

In a preferred embodiment, the curve fitting stops if the MSE<0.00010. The parameters are determined to have stopped changing when the L2-Norm of the parameter vector has not changed by more than 0.02510 from the last iteration. In a preferred embodiment, the L2-Norm of a vector is defined in the standard manner (NornParam) for the parameter vector of length NumParams values:

$$L2 - Norm = \sqrt{{\sum }_{i = 0}^{NumParams - 1} {( {Parameter}_{i} )}^{2}}$$

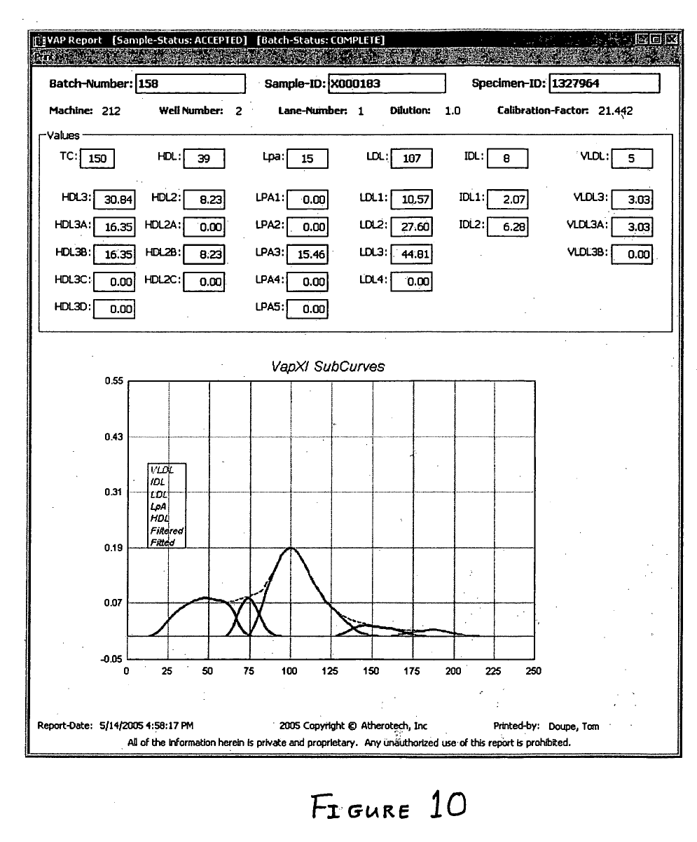

If the stop conditions have been met, then the cholesterol class values based on the profile (calculated using the optimization) is output is step 132 and the process is completed. FIG. 10 is an example of the user interface that shows the calculated cholesterol class values in accordance with the invention.

FIGS. 4A and 4B summarize the calculations in a flow chart. In step 134, an optimal step change vector is computed. In step 136, the profile shape based on the updated parameters (due to the step change vector) is computed. In step 138, the software executing on the computer system calculates the error between the newly computed profile and the profile generated by the spectrometer and then compares the error to determine if it has been reduced based on the computed profile shape. If the error has been reduced, then the damping is decreased and the parameter estimates for both constrained and unconstrained parameters are updated in step 140 and the method loops back to step 130. If the error has not been reduced, then in step 142, the last set of computed parameters are ignored, the damping factor is increased and a new step size is selected and the method loops back to step 130. In this manner, the values of the cholesterol classes are determined based on the absorption profile.

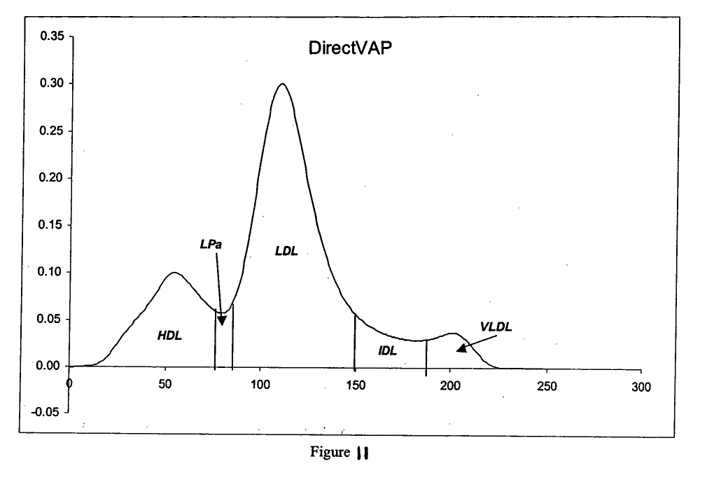

As discussed above, the dynamic initialization may also permit a different cholesterol class value determination to be used that does not use subcurves or curve fitting methods as described above. In particular, the cholesterol determining method may use a direct area measurement that is independent of any subcurve shape or assumed hydrodynamic conditions. In particular, since dynamic initialization is used, it is possible to measure the cholesterol subclasses by simply defining area boundaries based on the feature locations which would require no iterations or curve fitting. Thus, after the feature detection as described above, the appropriate subareas are defined and the integral of the profile within that boundary could be readily measured. An example profile with the cholesterol class values computed in this manner is shown in FIG. 11.

Although the preferred embodiment described above is directed to a particular profile generation technique and the determination of cholesterol classes and subclasses, the invention has broader applicability to any physiological parameter that may include cholesterol, but may also include other physiological parameters such as protein concentrations, glucose concentrations, cellular type, etc. When the above described system and method are used to determine another type of physiological parameter, the number of subcurves, subcurve parameters, reagent and process settings are adapted as is known in the art. Furthermore, as one skilled in the art will recognize, different process settings, sample rates and/or hardware configurations will require different starting and allowable ranges that are within the scope of the invention. Similarly, a change in the number of subcurves could be easily managed by the present invention with suitable alteration of the starting values and allowable ranges. For example, if the invention is used to determine lipoproteins, the various constants and variable values described above for the cholesterol subclass determination would need to be changed as would be understood by one or ordinary skill in the art.

In addition, the invention may be used when other techniques are utilized to generate the profile. These techniques may include manual fluid dispersion and manual or automated scanning of photos or actual gel stains. For example, for a lipoprotein class determination, the invention may be used with a system in which the profile of the lipoproteins is prepared using ultracentrifuge wherein blood serum or plasma is stained and then the stained plasma or serum is added to a density gradient forming solute solution wherein the gradients are formed when a centrifugal force is applied to the combination of the stained plasma or serum and the density gradient forming solute solution. Further details of this technique for forming the profile for a lipoprotein is described in U.S. Pat. No. 6,753,185 which is incorporated herein by reference.

While the foregoing has been-with reference to a particular embodiment of the invention, it will be appreciated by those skilled in the art that changes in this embodiment may be made without departing from the principles and spirit of the invention, the scope of which is defined by the appended claims.

1. A system for determining a set of physiological parameter basis class values, comprising:
a unit that generates a profile based on a physiological parameter sample; and
a computer system that analyzes the profile and generates a set of physiological parameter class values based on the profile, the computer system having means for dynamically initializing a set of parameters to generate a set of initial parameters and means for determining a set of physiological parameter values based on the set of parameters and the profile.

2. The system of claim 1, wherein the physiological parameter further comprise one of cholesterol, lipoproteins, protein concentration, glucose concentration and cellular type.

3. The system of claim 2, wherein the determining means further comprising means for fitting a set of predetermined subcurves to the profile wherein each physiological parameter class has a subcurve.

4. The system of claim 3, wherein the fitting means further comprising means for using a Levenberg Marquardt optimization.

5. The system of claim 4, wherein the optimization means further comprises means for comparing a calculated curve point to a fitted curve point to generate an error function, means for adjusting the fitting function based on the error function.

6. The system of claim 3, wherein the fitting means further comprising means for determining a particular subcurve for a particular physiological parameter class based on a Weibull equation.

7. The system of claim 4, wherein the fitting means further comprising means for determining a particular subcurve for a particular physiological parameter class based on a Weibull equation.

8. The system of claim 1, wherein the determining means further comprising means for performing a direct area measurement of an area under the profile in order to determine each physiological parameter class value.

9. The system of claim 2, wherein the unit further comprises a spectrometer that generates an absorption profile based on the physiological parameter sample.

10. A system for determining a set of cholesterol class values, comprising:
a detector that generates a value corresponding to a cholesterol sample; and
a computer system that analyzes the cholesterol sample value and generates a set of cholesterol class values based on the cholesterol sample value, the computer system having means for dynamically initializing a set of parameters to generate a set of initial parameters and means for determining a set of cholesterol values based on the set of parameters and the cholesterol sample value.

11. The system of claim 10, wherein the determining means further comprising means for fitting a set of predetermined subcurves to the cholesterol sample value wherein each cholesterol class has a subcurve.

12. The system of claim 11, wherein the fitting means further comprising means for using a Levenberg Marquardt optimization.

13. The system of claim 12, wherein the optimization means further comprises means for comparing a calculated curve point to a fitted curve point to generate an error function, means for adjusting the fitting function based on the error function.

14. The system of claim 11, wherein the fitting means further comprising means for determining a particular subcurve for a particular cholesterol class based on a Weibull equation.

15. The system of claim 12, wherein the fitting means further comprising means for determining a particular subcurve for a particular cholesterol class based on a Weibull equation.

16. A computer implemented method for determining a set of physiological parameter class values, comprising:
determining a value corresponding to a physiological parameter sample;
analyzing the physiological parameter sample value to generate a set of physiological parameter class values based on the physiological parameter sample value; and
wherein the analyzing further comprises dynamically initializing a set of parameters to generate a set of initial parameters and determining a set of physiological parameter values based on the set of parameters and the physiological parameter sample value.

17. The method of claim 16, wherein the physiological parameter further comprise one of cholesterol, lipoproteins, protein concentration, glucose concentration and cellular type..

18. The method of claim 17, wherein determining the set of physiological parameter values further comprises fitting a set of predetermined subcurves to the profile wherein each physiological parameter class has a subcurve.

19. The method of claim 18, wherein the fitting further comprising using a Levenberg Marquardt optimization.

20. The method of claim 19, wherein the fitting further comprises comparing a calculated curve point to a fitted curve point to generate an error function and adjusting the fitting function based on the error function.

21. The method of claim 18, wherein the fitting further comprising determining a particular subcurve for a particular physiological parameter class based on a Weibull equation.

22. The method of claim 19, wherein the fitting further comprising determining a particular subcurve for a particular physiological parameter class based on a Weibull equation.

23. The method of claim 17, wherein the determining the set of physiological parameter values further comprises performing a direct area measurement of an area under the profile in order to determine each physiological parameter class value.

24. A computer implemented method for determining a set of cholesterol class values, comprising:
determining a value corresponding to a cholesterol sample;
analyzing the cholesterol sample value to generate a set of cholesterol class values based on the cholesterol sample value; and
wherein the analyzing further comprises dynamically initializing a set of parameters to generate a set of initial parameters and determining a set of cholesterol values based on the set of parameters and the cholesterol sample value.

25. The method of claim 24, wherein the determining further comprises fitting a set of predetermined subcurves to the cholesterol sample value wherein each cholesterol class has a subcurve.

26. The method of claim 25, wherein the fitting further comprising using a Levenberg Marquardt optimization.

27. The method of claim 26, wherein the optimization further comprises comparing a calculated curve point to a fitted curve point to generate an error function and adjusting an error function based on the error function.

28. The method of claim 25, wherein the fitting further comprising determining a particular subcurve for a particular cholesterol class based on a Weibull equation.

29. The method of claim 26, wherein the fitting further comprising determining a particular subcurve for a particular cholesterol class based on a Weibull equation.

30. A system for determining a set of cholesterol basis class values, comprising:
a detector that generates an absorption profile based on a cholesterol sample; and
a computer system that analyzes the absorption profile and generates a set of cholesterol class values based on the absorption profile, the computer system having means for dynamically initializing a set of parameters to generate a set of initial parameters and means for determining a set of cholesterol values based on the set of parameters and the absorption profile.

31. The system of claim 30, wherein the determining means further comprising means for fitting a set of predetermined subcurves to the absorption profile wherein each cholesterol class has a subcurve.

32. The system of claim 31, wherein the fitting means further comprising means for using a Levenberg Marquardt optimization.

33. The system of claim 32, wherein the optimization means further comprises means for comparing a calculated curve point to a fitted curve point to generate an error function, means for adjusting the fitting function based on the error function.

34. The system of claim 31, wherein the fitting means further comprising means for determining a particular subcurve for a particular cholesterol class based on a Weibull equation.

35. The system of claim 32, wherein the fitting means further comprising means for determining a particular subcurve for a particular cholesterol class based on a Weibull equation.

36. The system of claim 30, wherein the determining means further comprising means for performing a direct area measurement of an area under the absorption profile in order to determine each cholesterol class value.

37. The system of claim 30, wherein the detector further comprises a spectrometer.
